(19) United States
(12) Patent Application Publication
(10) Pub. No.: US 2025/0152836 A1
(43) Pub. Date: May 15, 2025

(54) A sub-assembly for a medicament delivery device

(71) Applicant: SHL Medical AG, Zug (CH)

(72) Inventor: Anders Boström, Nacka Strand (SE)

(21) Appl. No.: 18/832,770

(22) PCT Filed: Jan. 10, 2023
(86) PCT No.: PCT/EP2023/050369

§ 371 Date: Jul. 24, 2024

(30) Foreign Application Priority Data
Jan. 24, 2022 (EP) 22152917.5

(51) Int. Cl. A61M 5/315; A61M 5/20

(52) U.S. Cl. CPC A61M 5/3157; A61M 5/2033; A61M 5/31511; A61M 2205/581; A61M 2205/582

(57) ABSTRACT

The present disclosure generally relates to medicament delivery devices such as autoinjectors, and particularly concerns a sub-assembly adapted to provide a plunger rod release function.

### CROSS-REFERENCE TO RELATED APPLICATIONS

The present application is a U.S. National Phase application pursuant to 35 U.S.C. § 371 of International Application No. PCT/EP2023/050369 filed Jan. 10, 2023, which claims priority to European Patent Application No. 22152917.5 filed Jan. 24, 2022. The entire disclosure contents of these applications are herewith incorporated by reference into the present application.

### TECHNICAL FIELD

The present disclosure generally relates to medicament delivery devices such as autoinjectors, and particularly concerns a sub-assembly adapted to provide a plunger rod release function.

### BACKGROUND

A number of medical conditions require injections. These days, a number of different injection devices exist, including various types of pen injectors, autoinjectors and on-body devices. Although many of these devices have enabled major improvements in the management of a number of medical conditions, various limitations do still exist in the current technology. Not least amongst these are the difficulties faced by patients that require frequent injections and by patients that need to inject particularly viscous drugs. In considering these problems, the applicant has appreciated that various developments could be made to help improve the medicament delivery devices on the market today, for example concerning the force of injection by a plunger, which are set out in more detail below.

### SUMMARY

An object of the present disclosure is to provide a sub-assembly for a medicament delivery device which solves, or at least mitigates problems of the prior art.

According to a first aspect of the present disclosure, there is provided a sub-assembly of a medicament delivery device.

The sub-assembly comprising a housing extending along a longitudinal axis between a distal end and a proximal end, wherein the housing comprises a protrusion extending in a direction transverse to the longitudinal axis, wherein the protrusion comprises a distally directed surface;
an actuator comprising a support wall extending towards the proximal end of the housing;
a plunger rod comprising a flexible arm extending towards the distal end of the housing; wherein the flexible arm comprises a protrusion extending radially outwardly relative to the longitudinal axis, wherein the protrusion comprises a proximally directed surface adjacent to the distally directed surface of the housing, the flexible arm is arranged between the support wall of the actuator and an inner wall of the housing in a direction transverse to the longitudinal axis;
a biasing member extending in the direction of the longitudinal axis between a proximally directed surface of the housing and a distally directed surface of the plunger rod; and
wherein the actuator is axially moveable relative to the housing between a proximal position where the support wall is adjacent to an inner surface of the flexible arm of the plunger rod and a distal position where the support wall is axially offset to the inner surface of the flexible arm.

Embodiments of the present disclosure advantageously provides maintaining the plunger rod in its initial position while the actuator is in the proximal position, and once the actuator is moved to the distal position the plunger rod is released to add force to the injection by means of the biasing member that pushes the plunger rod in the proximal direction. When the actuator is in its proximal position, the support wall reaches to a position so that the flexible arm is interleaved between the support wall of the actuator and the protrusion of the housing. In this position the flexible arm is not allowed to flex due to that the protrusion of the flexible arm is hindered by the support wall of the actuator and the protrusion of the housing. However, once the actuator has moved to the distal position, the support wall has moved away axially from the flexible arm, whereby the flexible arm can flex radially outwards under force of the biasing member, and the plunger rod is released for the injection.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

When the wording ‘at the injection site’ or ‘at the dose delivery site’ is used in this application, it generally refers to the point where the medicament delivery device (e.g., a needle) enters the patient, along with the surrounding area, for example the area where a pad is attached.

According to one embodiment, the housing may comprise at least two protrusions, wherein a distal opening of the housing is defined between the two protrusions. The at least two protrusions may be symmetrically arranged about the distal opening. Having more than one protrusion distributed about the opening provides for improved support for the protrusion of the flexible arm when the actuator is in the proximal position to better maintain the plunger rod in its initial position before injection.

According to one embodiment, the protrusion of the housing may be an annular protrusion, wherein a distal opening of the housing is defined in a surrounded center of the annular protrusion. The annular protrusion provides for improved support for the protrusion of the flexible arm when the actuator is in the proximal position.

According to one embodiment, the distal directed surface is defined on an inner edge of the annular protrusion.

According to one embodiment, wherein the flexible arm of the plunger rod protrudes through the distal opening of the housing. Thus, the flexible arm extends beyond the distal surface of the housing in the distal direction.

According to one embodiment, the inner wall extends from the distal opening towards the proximal end of the housing, wherein the protrusion extends from the inner wall of the housing to an outer wall of the housing.

According to one embodiment, the flexible arm of the plunger rod may extend along the inner wall of the housing towards the distal end of the housing. The inner wall may advantageously provide a support for the flexible arms.

According to one embodiment, the inner wall of the housing may comprise a click element, wherein the flexible arm of the plunger rod comprises a counter click element configured to interact with the click element of the housing to generate an audible and/or tactile indication. This advantageously provides for providing an indication to a user of a successful release of the plunger rod.

According to one embodiment, the click element of the housing may be a protrusion facing towards the longitudinal axis.

According to one embodiment, the inner wall of the housing may comprise a flexible tab, wherein the click element of the housing is arranged on the flexible tab. A flexible tab provides a relatively simple yet robust way to enable an audible and/or tactile indication.

According to one embodiment, the protrusion of the plunger rod may advantageously comprise the counter click element.

According to one embodiment, the click element of the plunger rod may be a cut-out/recess in a sidewall of the flexible arm. This provides another relatively simple yet robust way to enable an audible and/or tactile indication.

According to one embodiment, the protrusion of the flexible arm of the plunger rod may extend from a distal end of the flexible arm.

According to one embodiment, wherein the sub-assembly comprises a delivery member cover axially movable relative to the housing; wherein the delivery member cover comprises a tubular section configured to surround a medicament delivery member of the medicament delivery device; wherein the actuator extends from the tubular section towards the distal end of the housing. The tubular section abuts on the actuator when the cover is moved in a distal direction. In this way, the delivery member cover apply force on actuator so that the actuator moves axially.

According to one embodiment, at least one of the distally directed surface of the housing and the proximally directed surface of the plunger rod may be bevelled. This provides for a more efficient force direction for flexing the flexible arms towards the longitudinal axis.

There is further provided a medicament delivery device comprising the sub-assembly of any of the herein disclosed embodiments.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 is a perspective view of an autoinjector according to embodiments of the present disclosure;

FIG. 2A illustrates medicament delivery device with the front cap removed according to embodiments of the present disclosure;

FIG. 2B illustrates medicament delivery device with the delivery member cover in a retracted position according to embodiments of the present disclosure;

FIG. 2C illustrates medicament delivery device with the delivery member cover in an extended position after a medicament delivery operation according to embodiments of the present disclosure;

FIG. 3 is an exploded view of a sub-assembly according to embodiments of the present disclosure;

FIG. 4A-C are different perspective views of a housing according to embodiments of the present disclosure;

FIG. 5 is a perspective view of a plunger rod according to embodiments of the present disclosure;

FIG. 6 is a perspective view of an actuator according to embodiments of the present disclosure;

FIG. 7 is a perspective view of a delivery member cover according to embodiments of the present disclosure;

FIG. 8 is a cross-section of the sub-assembly in an initial state before injection with the actuator in a proximal position according to embodiments of the present disclosure;

FIG. 9 is a cross-section of the sub-assembly during an activation stroke of the delivery member cover according to embodiments of the present disclosure;

FIG. 10 is a cross-section of the sub-assembly when the actuator is in the distal position according to embodiments of the present disclosure;

FIG. 11 is a cross-section of the sub-assembly after an injection is finished according to embodiments of the present disclosure;

FIG. 12 is a cross-sectional view of another embodiment of a sub-assembly of the present disclosure before injection; and

FIG. 13 is a cross-sectional view of another embodiment of a sub-assembly of the present disclosure after injection.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

In the Figures, the longitudinal direction is the direction of axis 102, with the corresponding circumferential direction 31 and radial direction 33 relative to the axis 102 also shown.

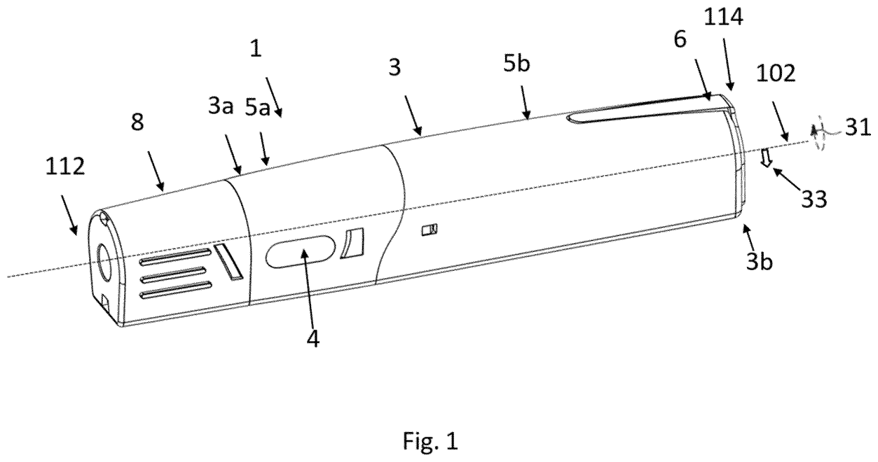

FIG. 1 shows an example of a medicament delivery device 1 such as an autoinjector according to embodiments of the present disclosure. The medicament delivery device 1 is configured to expel medicament from a medicament container via a medicament delivery device to a user at a dose delivery site. The medicament delivery device 1 extends from a proximal end 112 to a distal end 114 relative to the axis 102.

The medicament delivery device 1 comprises a housing 3 with a window 4. The housing 3 has a proximal end 3a and a distal end 3b, and a proximal housing part 5a and a distal housing part 5b. A safety clip 6 is arranged at the distal end 3b of the housing and is removed before use of the medicament delivery device 1. A front cap 8 is arranged at proximal end 3a of the housing to cover a delivery member cover.

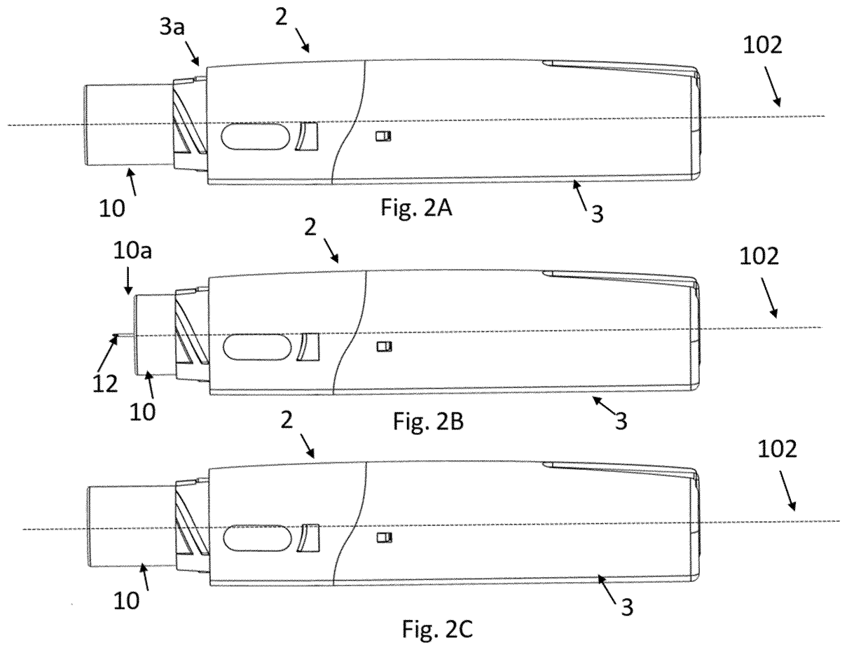

FIG. 2A illustrates the medicament delivery device with the front cap 8 removed so that the delivery member cover 10 is exposed. The delivery member cover 10 is configured to surround a medicament delivery member, such as a needle 12 at the proximal end 3a of the housing. The extended position is considered a proximal position of the delivery member cover 10. The delivery member cover 10 is biased by e.g., a spring in the proximal direction towards the extended position.

In FIG. 2B, the delivery member cover 10 has been axially moved in relation to the housing 3 along the longitudinal axis 102 to a retracted position where the delivery member cover 10 is received further in the housing 3 and in which the needle 12 is exposed at the proximal end 10a of the delivery member cover 10. In this position of the delivery member cover 10 a plunger rod, discussed with reference to subsequent drawings, causes expulsion of medicament from the medicament container by a force generated by for example a spring that acts on the plunger rod.

After a medicament delivery operation, the delivery member cover 10 is returned turn the extended position, again shown in FIG. 2C, to thereby cover the needle 12 so that a sharp injury can be prevented.

It would be desirable to increase the force of injection provided by the plunger rod. To achieve this, the present disclosure discloses a sub-assembly that will now be described in more detail with reference to subsequent drawings.

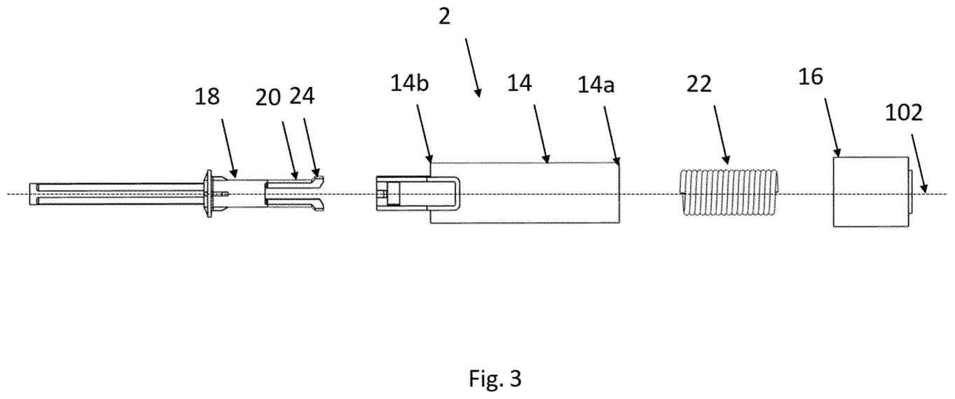

FIG. 3 is an exploded view of sub-assembly 2 which comprises a housing 14, an actuator 16, a plunger rod 18 comprising a flexible arm 20, and a biasing member 22 in the form of a spring.

The plunger rod 18 and the spring 22 is arranged inside the housing 14, and the actuator is arranged at the distal end 14a of the housing 14. The plunger rod 18 is axially displaceable in the housing 14 during an injection phase of the sub-assembly 2. The housing 14 is fixed in relation to the outermost housing 3 shown in the preceding drawings. The separate parts of the sub-assembly 2 will now be describe in more detail.

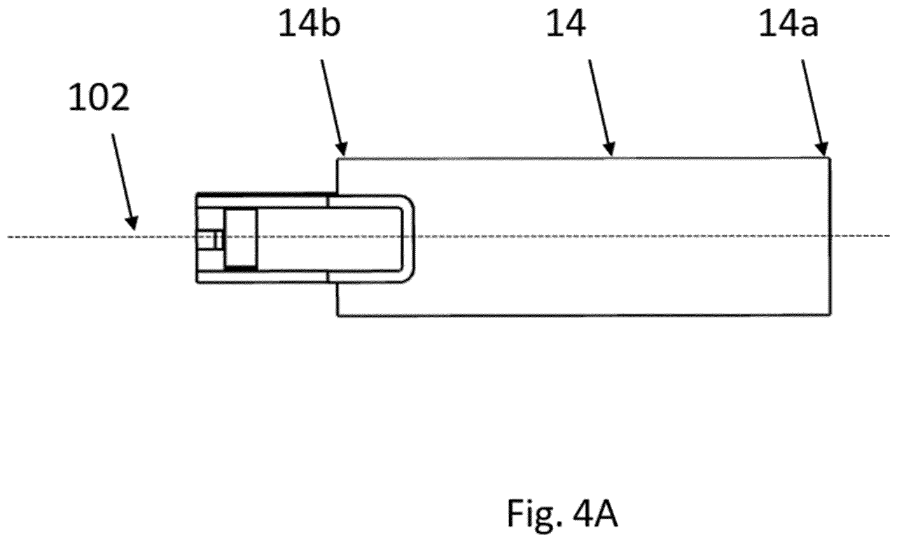

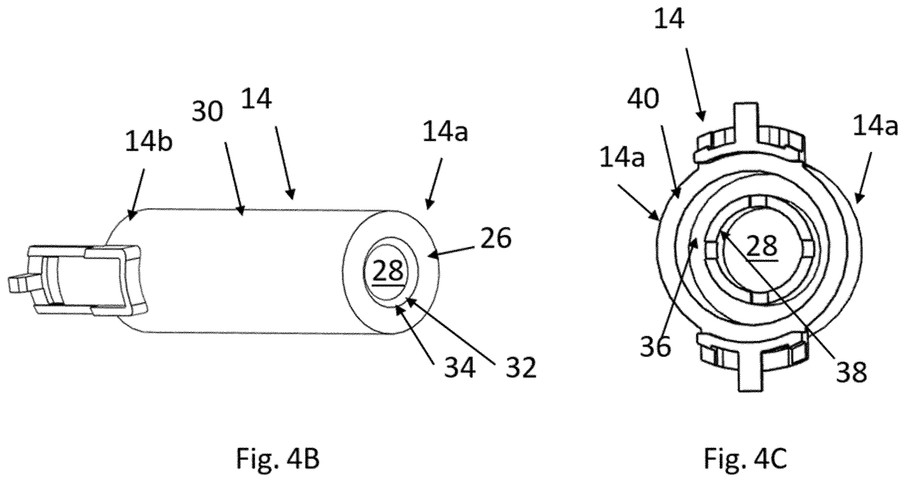

Turning to FIGS. 4A-B, illustrating two different views of the housing 14 having a generally cylindrical main body 30. The housing 14 extends along the longitudinal axis 102 between the distal end 14a and a proximal end 14b. Further, as is better seen in FIG. 4B the housing 14 comprises a protrusion 26 extending in a direction transverse to the longitudinal axis 102. In this example embodiment, the protrusion 26 is an annular protrusion 26. A distal opening 28 of the housing is defined in a surrounded centre of the annular protrusion 26. The distal opening is a through-hole extending along the longitudinal axis 102 and in which the plunger rod 18 may be arranged. The opening 28 may be an annular opening.

In other possible embodiments, instead of an annular protrusion, at least two protrusions may be used. In such case, the distal opening of the housing is defined between the two protrusions.

Turning now to FIG. 4B, the protrusion 26 comprises a distally directed surface 32. In this embodiment, the distally directed surface 32 is an annular surface that is coaxial with and surrounds the distal opening 28.

More specifically, the distal directed surface 32 is defined on an inner edge 34 of the annular protrusion 26. The inner edge 34 is the portion of the annular protrusion 26 that is closest to the longitudinal axis 102. In other words, the inner edge 34 is the radially innermost portion of the annular protrusion 26.

Further, as is best seen in FIG. 4C, the protrusion 26 comprises a proximally directed surface 36 for receiving the distal end of the biasing member 22. The proximally directed surface is an annular surface that surrounds the distal opening 28. The proximally directed surface 36 is sufficiently wide to receive the spring 22. Further, the shape of the proximally directed surface 36 extension substantially matches the annular shape of the spring 22.

The distally directed surface 32 of the housing 14 is bevelled. In other words, the distally directed surface 32 is inclined with respect to an axis perpendicular to the longitudinal axis 102.

Further, the housing comprises an inner wall 38 that is cylindrical in shape. The inner wall 38 extends from the distal opening 28 towards the proximal end of the housing 14. The protrusion 26 extends from the inner wall 38 of the housing to an outer wall 40 of the housing 14. The inner wall 38 and the outer wall 40 are cylindrical and coaxially arranged. The inner wall 38 and the outer wall 40 are and connected through the protrusion 26. Further, the space between the inner wall 38 and the outer wall 40, where the proximally directed surface is located, is configured so that the spring can be installed in the space between the inner wall 38 and the outer wall 40 of the housing coaxial with the inner wall 38 and the outer wall 40.

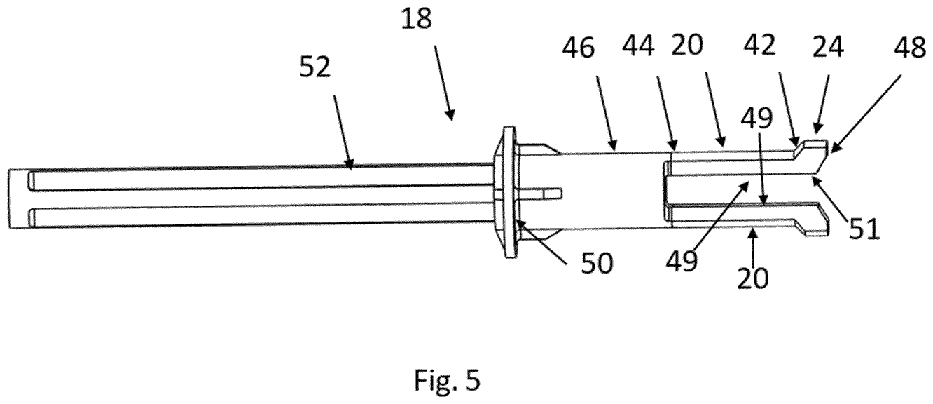

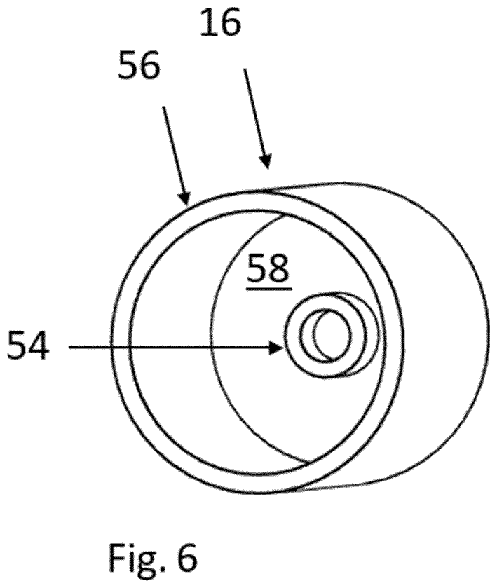

FIG. 5 is a perspective view of the plunger rod 18. The plunger rod 18 comprising a flexible arm 20 extending towards the distal end of the housing, see also FIG. 3. The flexible arm 20 comprises a protrusion 24 extending radially outwardly relative to the longitudinal axis. Further, the protrusion 20 comprises a proximally directed surface 42 which is adapted to be adjacent to the distally directed surface 32 of the housing 14. The proximally directed surface 42 of the plunger rod 18 is bevelled. In other words, the distally directed surface 32 is inclined with respect to an axis perpendicular to the longitudinal axis 102.

The flexible arm 20 is flexible so that the protrusion 24 is movable towards the longitudinal axis 102 by a flexing motion of the flexible arm 20. The flexible arm 20 comprises a fixed end 44 connected to a main body 46 of the plunger rod 18 and a free end 48 movable in relation to the fixed end 44. The fixed end 44 is a proximal end of the flexible arm 20 and the free end 48 is a distal end 51 of the flexible arm from which the protrusion 24 extends in the radial direction.

The plunger rod 18 may comprise more than one flexible arm 20 and in this example embodiment two flexible arm 20 are shown. The flexible arms 20 are symmetrically arranged on opposite sides of the longitudinal axis 102.

The plunger rod 18 further comprises a distally directed surface 50 for receiving the proximal end of the biasing member 22. On a proximal end of the plunger rod 18 is a rod 52 arranged that transfers an injection force form the biasing member 22 to the medicament container.

The flexible arms 20 of the plunger rod 18 each comprises an inner surface 49 directed towards the longitudinal axis 102 of the housing 14.

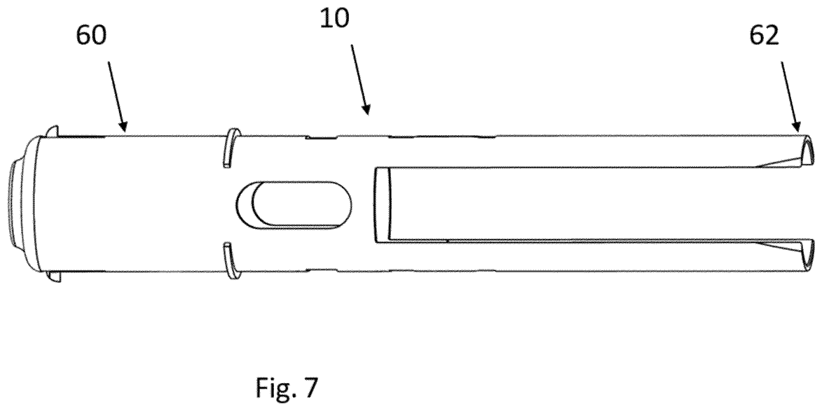

FIG. 6 is a perspective view of the actuator 16. The actuator 16 comprises a support wall 54 that, when arranged in the sub-assembly, extends towards the proximal end 14b of the housing 14. The support wall 54 is cylindrical and is coaxial with an outer wall 56 of the actuator 16. The diameter of the outer wall 56 is larger than the diameter of the support wall 54. The support wall 54 and the outer wall are connected through a distal plate structure 58.

In some embodiments, the sub-assembly comprises the delivery member cover 10 that is axially movable relative to the housing 14.

FIG. 7 is a perspective view of one such delivery member cover 10, which was also described with reference to FIGS. 2A-C. The delivery member cover 10 comprises a proximal tubular section 60 configured to cover a needle. The delivery member cover 10 further comprises a distal end 62 that extends towards the actuator 16 and the distal end 14a of the housing 14.

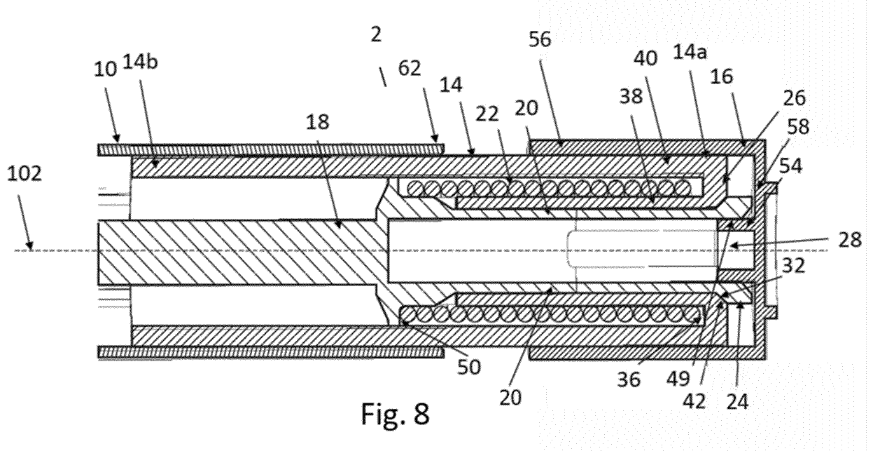

FIG. 8 is a cross-section of the sub-assembly 2 in an initial state. Here, the actuator 16 is in a proximal position where the support wall 54 of the actuator 16 is adjacent to an inner surface 49 of the flexible arm 20 of the plunger rod 18.

Here, the flexible arms 20 are arranged between the support wall 54 of the actuator 16 and an inner wall 38 of the housing in a direction transverse to the longitudinal axis 102. In particular, the support wall 54 is adjacent to an inner surface 49 of the flexible arm 20 of the plunger rod 18.

The spring 22 is compressed between the proximally directed surface 36 of the housing 14 and the distally directed surface 50 of the plunger rod 18. The spring 22 is adapted to apply a force on the distal surface 50 of the plunger rod 18 to axially move the plunger rod 18 in the proximal direction in relation to the housing 14. However, in this state, the protrusions 24 of the flexible arms 20 are prevented from moving radially inwards towards the longitudinal axis 102 by the annular protrusion 26 and the support wall 54 of the actuator 16 that extends from the distal plate 58 towards the proximal end 14b of the housing 14. Thus, the flexible arms 20 must flex radially inwards to allow for the protrusions to fit and move into the annular opening 28 of the housing 14.

In other words, in this state shown in FIG. 8, the proximally directed surface 42 of the protrusion 24 and the distally directed surface 32 of the protrusion 26 are in contact and do not move substantially with respect to each other since the support wall 54 prevents the radial inwards flexing motion of the flexible arms 20.

To facilitate that the plunger rod is kept in place, the flexible arm 20 of the plunger rod 18 protrudes through the distal opening 28 of the housing 14.

As briefly discussed in relation to FIGS. 4A-C the housing 14 comprises an inner wall 38 extending from the distal opening 28 towards the proximal end 14b of the housing 14. The protrusion 26 extends from the inner wall 38 of the housing to an outer wall 40 of the housing 14. The spring 22 is accommodated between the inner wall 38 and the outer wall 40 of the housing 14.

The flexible arms 20 of the plunger rod 18 extend along the inner wall 38 of the housing towards the distal end 14a of the housing 14.

The delivery member cover 10 which is axially movable relative to the housing 14 includes a distal end 62. The housing 14 is at least partly arranged inside the delivery member cover 10, here the proximal end of the housing 14 is inside the delivery member cover 10.

The actuator 16 is axially movable with respect to the housing 14 and comprises an outer wall 56. The distal end 62 of the delivery member cover 10 extends from a tubular section 60 of the delivery member cover 10 towards the actuator 16. More specifically, the distal end 62 of the delivery member cover 10 extends towards the proximal end of the outer wall 56 of the actuator.

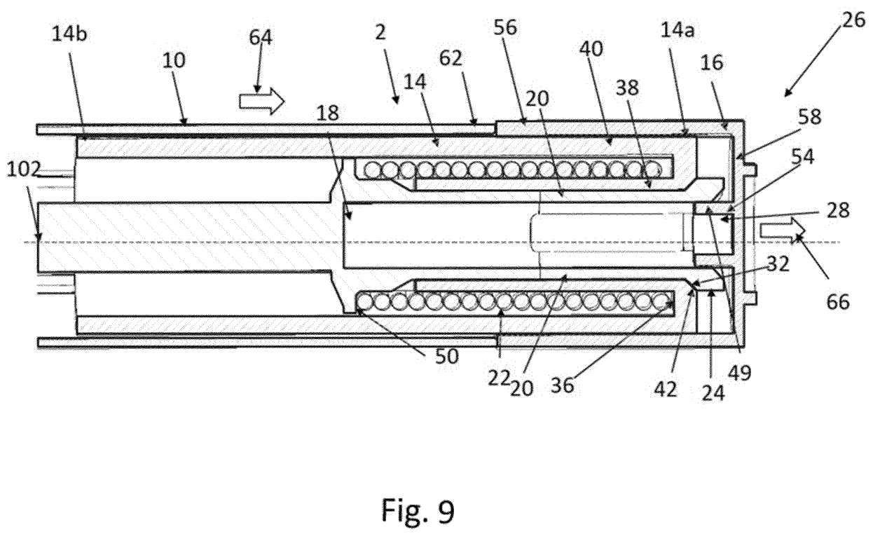

Turning now to FIG. 9, where, during an activation stroke of the delivery member cover 10, the delivery cover structure 10 has moved linearly in the distal direction as indicated by arrow 64 to expose the needle, see FIG. 2B. The distal end 62 of the medicament delivery member 10 abuts on the outer wall 56 of the actuator 16. The actuator 16 is axially movable in the distal direction and is caused by the action of the delivery member cover 10 to move in the distal direction as indicated by arrow 66. The support wall 54 of the actuator is still adjacent to an inner surface 49 of the flexible arm of the plunger rod 18.

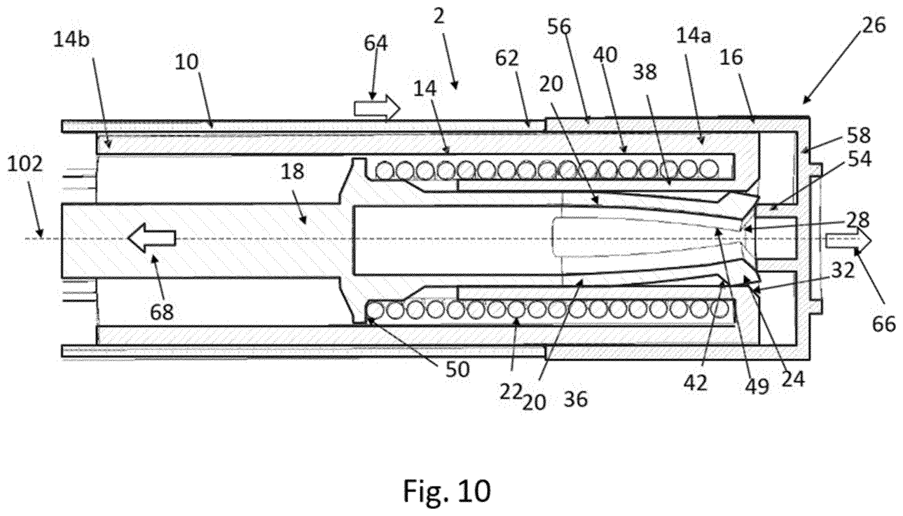

Turning now to FIG. 10, where the actuator 16 has moved to a distal position where the support wall 54 is axially offset to the inner surface 49 of the flexible arms 20. The support wall 54 and the flexible arms are no longer in contact with each other.

The interaction between the bevelled proximal surface 42 of the flexible arm 20 and the bevelled surface 32 of the protrusion 26 of the housing 14, under the action of the spring 22, forces the flexible arms to flex radially inwards towards the longitudinal axis 102 and the plunger rod moves in the proximal direction indicated by arrow 68 under the action of the spring 22 The flexible arms 20 move through the opening 28 and into the housing 14.

When the actuator 16 is in this distal position, the plunger rod 18 is unlocked and can move in the proximal direction 68 under the influence of the spring 22 which pushes on the distally facing surface 50 of the plunger rod 18 by releasing its spring force.

As the spring 22 pushes on the distally facing surface 50 of the plunger rod 18 during injection, the plunger rod 18 applies an injection force for facilitating injection at a delivery site.

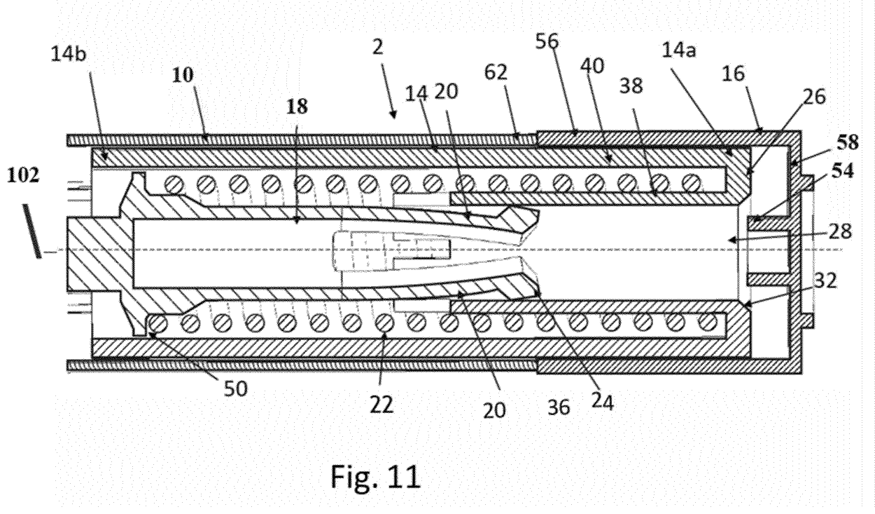

FIG. 11 illustrates the sub-assembly 2 after injection is finished. Here the flexible arms 20 are inside the inner wall 38 of the housing 14 after the plunger rod 18 has moved further in the proximal direction 68 under the influence of the spring 22.

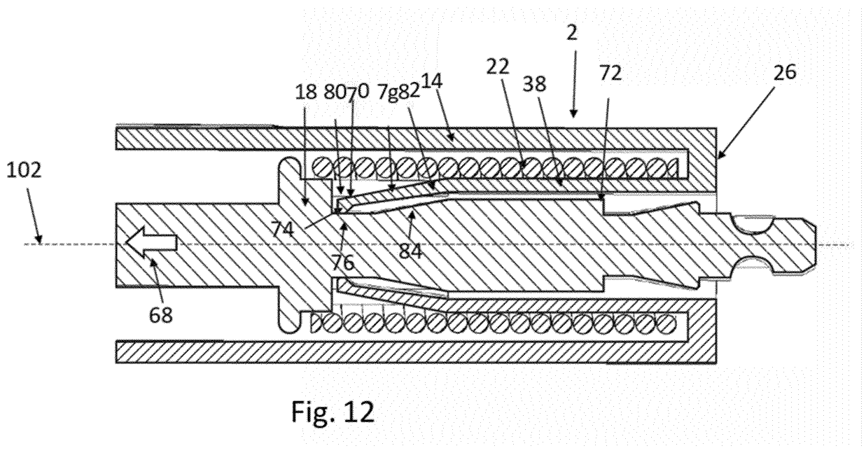

FIG. 12 is a cross-sectional view of another embodiment of a sub-assembly 2 of the present disclosure. Here, housing 14 comprises an additional element on the inner wall 38 in the form of a click element 70. The plunger rod 18 comprises a counter click element 72 configured to interact with the click element 70 of the housing 14 to generate an audible and/or tactile indication.

The counter click element 72 of the plunger rod 18 may be a separate element on the plunger rod, or it may be arranged on the flexible arm 20 described above in relation to preceding drawings. The audible/tactile indication is adapted to indicate to a user that injection using the medicament delivery device 1 is finished.

The click element 70 includes a protrusion 74 that faces towards the longitudinal axis 102, and thereby also towards the plunger rod 18 in the state shown in FIG. 12. The protrusion 74 is in this initial state, before injection, facing a first section 76 of the plunger rod 18.

The inner wall 38 of the housing 14 comprises a flexible tab 78. The flexible tab comprises a free proximal end 80 and a fixed distal end 82 attached to the inner wall 38 of the housing 14. This allows the free proximal end to flex radially with respect to the longitudinal axis 102 and with respect to the fixed end 82.

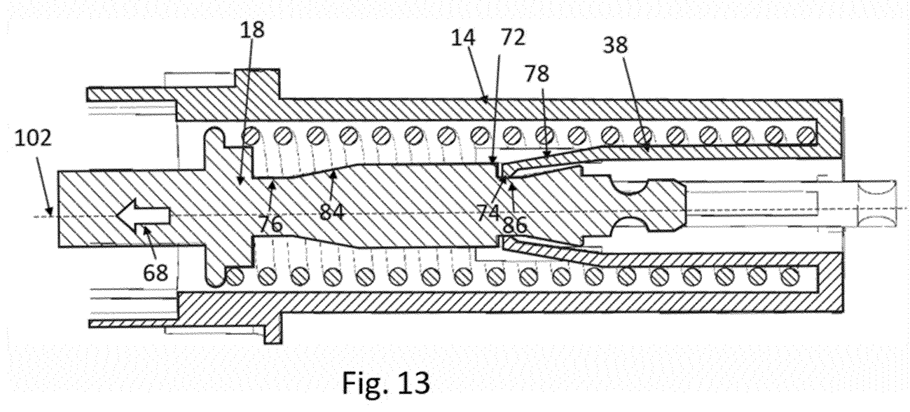

FIG. 13 illustrates the sub-assembly 200 after injection. As the plunger rod 18 moves in the proximal direction 68 with respect to the housing 14 the protrusion 74 slides along the surface of the plunger rod 18 on the ramp surface 84 which pushes the flexible tab 78 radially outwards with respect to the longitudinal axis 102. This biases the flexible tab which pushes back toward the longitudinal axis 102.

When the plunger rod 18 moves further in the proximal direction, the protrusion 74 moves to a position distal with respect to the counter click element 72 of the plunger rod 18. As the protrusion 74 moves passed counter click element 72 the flexible tab 78 flexes back and the protrusion 74 hits the surface of the plunger rod 18 in a second section 86 distally located with respect to the first section 76. As the click element 70 flexes onto the surface of the second section 86 an audible/tactile indication is generated.

In some embodiments, the protrusion 26 of the plunger rod 18 comprises the counter click element 70. Further, the click element of the plunger rod may be formed as a cut-out/recess in a sidewall of the flexible arm 20.

A medicament delivery device (such as an autoinjector) may generally include various other components. For example, a sensor unit which may recognize injection events, such as the autoinjector inserted into an attachment portion of e.g., a pad, injection started, and injection ends, a memory unit which is configured to store the recorded data during the injection, a connectivity unit configured to transmit the stored data to a smart device or the network directly, a processing unit configured to control the entire system and processes the data before transmitting it, and/or user interface units that are configured to provide feedback to the patient, such as status LEDs, haptic, and/or audio feedback.

When the medicament delivery device is placed into the attachment portion, the sensors inside of the support pad are configured to recognize the event and give feedback to the patient via haptic/visual or audio elements.

When the injection finishes, the sensors are configured to recognize the event and give feedback to the patient again. Further, the collected data is stored in the memory unit and may be transmitted to the smart device/network via the connectivity unit after the injection event finishes.

The sensor can be one of or the combination of the following: a mechanical switch, a Hall-effect sensor, an accelerometer.

The mechanical switch, hall-effect sensor, or accelerometer can be used for detection of the insertion of the auto-injector into an injection port.

The accelerometer can be used for detecting injection events.

Possible wireless communication methods include Bluetooth and Cellular Networks.

Bluetooth connectivity requires a smart device to transmit the stored data to the network and it requires a pairing action between the support pad and the smart device before being able to use the supporting pad. But it's a cheaper alternative and it requires less space on PCB.

The cellular network does not require any pairing process, it can be used as a plug-n-play device, no prior setup is needed, but it's more expensive and it requires more space on PCB.

Depending on the requirements of the product any of those two technologies can be used.

Such processing units may comprise a logic circuit or control unit including a microprocessor, microcontroller, programmable digital signal processor or another programmable device. The processing circuitry may also, or instead, each include an application specific integrated circuit, a programmable gate array or programmable array logic, a programmable logic device, or a digital signal processor. Where the processing circuitry includes a programmable device such as the microprocessor, microcontroller or programmable digital signal processor mentioned above, the processor may further include computer executable code that controls operation of the programmable device.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

The inventive concept has mainly been described above with reference to a few examples. However, as is readily appreciated by a person skilled in the art, other embodiments than the ones disclosed above are equally possible within the scope of the inventive concept, as defined by the appended claims.

1-18. (canceled)

19. A sub-assembly of a medicament delivery device, the sub-assembly comprising:
a housing extending along a longitudinal axis between a distal end and a proximal end, wherein the housing comprises a protrusion extending in a direction transverse to the longitudinal axis, wherein the protrusion comprises a distally directed surface;
an actuator comprising a support wall extending towards the proximal end of the housing;
a plunger rod comprising a flexible arm extending towards the distal end of the housing, wherein the flexible arm comprises a protrusion extending radially outwardly relative to the longitudinal axis, wherein the protrusion comprises a proximally directed surface adjacent to the distally directed surface of the housing, wherein the flexible arm is arranged between the support wall of the actuator and an inner wall of the housing in a direction transverse to the longitudinal axis; and
a biasing member extending in the direction of the longitudinal axis between a proximally directed surface of the housing and a distally directed surface of the plunger rod, wherein the actuator is axially moveable relative to the housing between a proximal position where the support wall is adjacent to an inner surface of the flexible arm of the plunger rod and a distal position where the support wall is axially offset to the inner surface of the flexible arm.

20. The sub-assembly according to claim 19, wherein the housing comprises at least two protrusions, and wherein a distal opening of the housing is defined between the two protrusions.

21. The sub-assembly according to claim 19, wherein the protrusion of the housing is an annular protrusion, and wherein a distal opening of the housing is defined in a surrounded center of the annular protrusion.

22. The sub-assembly according to claim 21, wherein the distal directed surface is defined on an inner edge of the annular protrusion.

23. The sub-assembly according to claim 20, wherein the flexible arm of the plunger rod protrudes through the distal opening of the housing.

24. The sub-assembly according to claim 20, wherein the inner wall extends from the distal opening towards the proximal end of the housing, wherein the protrusion extends from the inner wall of the housing to an outer wall of the housing.

25. The sub-assembly according to claim 24, wherein the flexible arm of the plunger rod extends along the inner wall of the housing towards the distal end of the housing.

26. The sub-assembly according to claim 25, wherein the inner wall of the housing comprises a click element, and wherein the flexible arm of the plunger rod comprises a counter click element configured to interact with the click element of the housing to generate an audible/tactile indication.

27. The sub-assembly according to claim 26, wherein the click element of the housing is a protrusion facing towards the longitudinal axis.

28. The sub-assembly according to claim 26, wherein the inner wall of the housing comprises a flexible tab, and wherein the click element of the housing is arranged on the flexible tab.

29. The sub-assembly according to claim 26, wherein the protrusion of the plunger rod comprises the counter click element.

30. The sub-assembly according to claim 26, wherein the click element of the plunger rod is a cut-out/recess in a sidewall of the flexible arm.

31. The sub-assembly according to claim 19, wherein the protrusion of the flexible arm of the plunger rod extends from a distal end of the flexible arm.

32. The sub-assembly according to claim 19, wherein the sub-assembly comprises a delivery member cover axially movable relative to the housing.

33. The sub-assembly according to claim 32, wherein the delivery member cover comprises a tubular section configured to surround a medicament delivery member of the medicament delivery device.

34. The sub-assembly according to claim 32, wherein the actuator extends from the tubular section towards the distal end of the housing.

35. The sub-assembly according to claim 32, wherein the sub-assembly is arranged so that, when the delivery member cover is moved in the distal direction relative to the housing, the delivery member cover abuts the actuator, thereby moving the actuator from the proximal position to the distal position, which allows the flexible arm to move towards the longitudinal axis, thereby releasing the plunger rod to be moved in the proximal direction by the biasing member.

36. The sub-assembly according to claim 19, wherein at least one of the distally directed surface of the housing and the proximally directed surface of the plunger rod is bevelled.
